(19) United States
(12) Patent Application Publication
(10) Pub. No.: US 2023/0174895 A1
(43) Pub. Date: Jun. 8, 2023

(54) GLYCEROL ACETAL SULFATE AND SULFONATE SURFACTANTS

(71) Applicant: The Procter & Gamble Company, Cincinnati, OH (US)

(72) Inventors: Rebecca Ann Langevin, Norwood, OH (US); Randall Thomas Reilman, Cincinnati, OH (US); Phillip Kyle Vinson, Fairfield, OH (US); John August Wos, Mason, OH (US); Robert Edward Shumate, Hamilton, OH (US)

(21) Appl. No.: 17/972,818

(22) Filed: Oct. 25, 2022

Related U.S. Application Data
(60) Provisional application No. 63/287,102, filed on Dec. 8, 2021.

(51) Int. Cl. C11D 1/83; C11D 11/00; C11D 1/825; C07D 321/00

(52) U.S. Cl. CPC C11D 1/83; C11D 11/0023; C11D 1/825; C07D 321/00; C11D 1/26

(57) ABSTRACT

Glycerol acetal sulfate and sulfonates, which may be used in consumer products to provide surfactant properties.

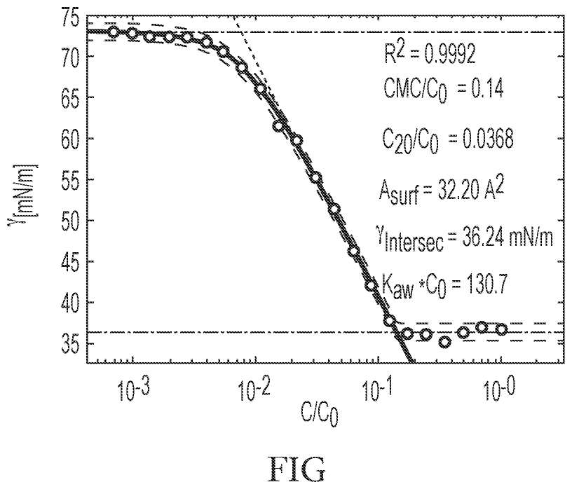

### FIELD OF THE INVENTION

Glycerol acetal sulfate and sulfonates, which may be used in consumer products to provide surfactant properties with excellent safety and environmental profiles.

### BACKGROUND OF THE INVENTION

Surfactants are widely used in everyday life in the form of consumer product compositions, such as detergents and shampoos to remove soils and provide the cleaning indicator of suds generation. There are many kinds of surfactants, and depending on their charge states at physiological pH, are classified as anionic surfactants, cationic surfactants, nonionic surfactants and amphoteric surfactants.

Alkyl and alkyl ether sulfates form, by far, the most popular group of anionic surfactants used as primary surfactants in shampoos. The alkyl and alkyl ether sulfates most widely used include sodium lauryl ether sulfate, sodium lauryl sulfate, ammonium lauryl ether sulfate, and ammonium lauryl sulfate. These are cost-effective materials that, if formulated effectively, deliver effective cleansing, foaming, rheology control and polymer deposition, but they have a disadvantage in that while they provide excellent washing power, they can cause strong skin irritation or leave hair brittle. To improve these deficiencies, a technique of adding hydrophilic ethylene oxide during the production of sodium laureth sulfate or ammonium laureth sulfate was developed to provide surfactant with sudsing properties, but reduced irritation as compared to conventional sodium lauryl sulfate or ammonium lauryl sulfate.

Anionic surfactants have been used, typically in combination with cosurfactants, especially amphoteric and zwitterionic co-surfactants such as amine oxide and betaines, to provide suds during dishwashing, with alkyl sulphate and alkyl alkoxy sulphates found to be particularly effective at providing improved sudsing in addition to the desired cleaning.

Ethoxylated surfactants are currently an anionic surfactant class heavily used for these purposes.

Accordingly, in order to solve the above problems, the present invention provides a surfactant prepared without the use of ethoxylation.

### SUMMARY OF THE INVENTION

The present invention relates to a compound comprising the following structure:

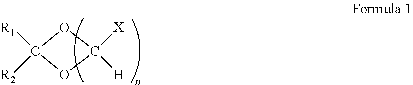

where:

n=2 or 3;

X=—H or —A-B such that only one carbon atom of the —(CH—X)ₙ— linking chain is attached to —A-B;

A=—O— or —CH₂—;

B=—SO₃⁻M⁺, where is M is a cation that forms an acceptable salt, —SO₃H, or —R₃ only when A=—O—;

R₁=a substituted or unsubstituted straight chain or branched chain alkyl or alkenyl, having 1 to 22 carbon atoms;

R2=H;

R3=hydroxyethyl, hydroxypropyl, methyl hydroxyethyl; including all possible stereoisomers thereof.

In some embodiments R1 has an average alkyl chain length of from 8 to 18, preferably from 10 to 14, more preferably from 12 to 14, most preferably from 12 to 13 carbon atoms. In preferred embodiments R1 is straight chain alkyl. In more preferred embodiments R1 is straight chain alkyl that is bio-based.

In some embodiments M is an alkali metal, an alkaline earth metal, ammonium, alkylammonium, or a mixture thereof. In embodiments M is an alkali metal, for example sodium.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

The FIGURE is a graph displaying critical micelle concentration (CMC) of a surfactant.

### DETAILED DESCRIPTION OF THE INVENTION

The present invention outlines a series of glycerol acetal sulfates and sulfonates (GAS) that can be used as replacements for existing ethoxylated surfactants.

The GAS's of the present invention may be used in liquid detergent compositions to provide a more consistent sudsing experience, regardless of the hardness of the water used to make the wash solution.

GAS's when used in liquid detergent compositions also provide the compositions with a good sudsing profile, including high initial suds volume generation and sustained suds stabilization through a dishwashing process, even when in presence of greasy and/or particulate soils, as well as good grease removal.

Definitions

As used herein, articles such as “a” and “an” when used in a claim, are understood to mean one or more of what is claimed or described.

The term “comprising” as used herein means that steps and ingredients other than those specifically mentioned can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “dishware” as used herein includes cookware and tableware made from, by non-limiting examples, ceramic, china, metal, glass, plastic (e.g., polyethylene, polypropylene, polystyrene, etc.) and wood.

The term “grease” or “greasy” as used herein means materials comprising at least in part (i.e., at least 0.5 wt % by weight of the grease) saturated and unsaturated fats and oils, preferably oils and fats derived from animal sources such as beef, pig and/or chicken.

The terms “include”, “includes” and “including” are meant to be non-limiting.

The term “particulate soils” as used herein means inorganic and especially organic, solid soil particles, especially food particles, such as for non-limiting examples: finely divided elemental carbon, baked grease particles, and meat particles.

The term “sudsing profile” as used herein refers to the properties of a detergent composition relating to suds character during the dishwashing process. The term “sudsing profile” of a detergent composition includes suds volume generated upon dissolving and agitation, typically manual agitation, of the detergent composition in the aqueous washing solution, and the retention of the suds during the dishwashing process. Preferably, hand dishwashing detergent compositions characterized as having “good sudsing profile” tend to have high suds volume and/or sustained suds volume, particularly during a substantial portion of or for the entire manual dishwashing process. This is important as the consumer uses high suds as an indicator that sufficient detergent composition has been dosed. Moreover, the consumer also uses the sustained suds volume as an indicator that sufficient active cleaning ingredients (e.g., surfactants) are present, even towards the end of the dishwashing process.

The consumer usually renews the washing solution when the sudsing subsides. Thus, a low sudsing detergent composition will tend to be replaced by the consumer more frequently than is necessary because of the low sudsing level.

The term “bio-based” material refers to a renewable material.

The term “renewable material” refers to a material that is produced from a renewable resource.

The term “renewable resource” refers to a resource that is produced via a natural process at a rate comparable to its rate of consumption (e.g., within a 100 year time frame). The resource can be replenished naturally, or via agricultural techniques. Non-limiting examples of renewable resources include plants (e.g., sugar cane, beets, corn, potatoes, citrus fruit, woody plants, lignocellulose, hemicellulose, cellulosic waste), animals, fish, bacteria, fungi, and forestry products. These resources can be naturally occurring, hybrids, or genetically engineered organisms. Natural resources, such as crude oil, coal, natural gas, and peat, which take longer than 100 years to form, are not considered renewable resources.

The term “bio-based content” refers to the amount of carbon from a renewable resource in a material as a percent of the weight (mass) of the total organic carbon in the material, as determined by ASTM D6866-10 Method B.

The term “petroleum-based” material refers to a material that is produced from fossil material, such as petroleum, natural gas, coal, etc.

A dash (-) preceding or following an atom or capital letter representing a chemical group indicates that the so-designated atom or chemical group has an open valence allowing it to connect to another so-designated atom or chemical group via a covalent bond. For example -A-B means that -A-B connects to another atom or group via a covalent bond from A to that other atom or group.

It is understood that the test methods that are disclosed in the Test Methods Section of the present application should be used to determine the respective values of the parameters of Applicants' inventions as described and claimed herein.

In all embodiments of the present invention, all percentages are by weight of the total composition, as evident by the context, unless specifically stated otherwise. All ratios are weight ratios, unless specifically stated otherwise, and all measurements are made at 25° C., unless otherwise designated.

Glycerol Acetal

Suitable glycerol acetal sulfate and sulfonate anionic surfactants have a structure according to formula

(I)

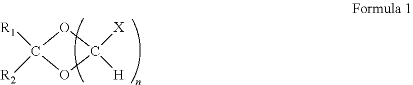

where:
n=2 or 3;
X=—H or —A-B such that only one carbon atom of the —(CH—X)ₙ— linking chain is attached to —A-B;
n=2 or 3, where the CH group is connected to A;
A=—O— or —CH₂—;
B=—SO₃⁻M⁺, where is M is a cation that forms an acceptable salt, —SO₃H, or —R₃ only when A=—O—;
R₁=a substituted or unsubstituted straight chain or branched chain alkyl or alkenyl, having 1 to 22 carbon atoms;
R2=H;
R3=hydroxyethyl, hydroxypropyl, methyl hydroxyethyl;
including all possible stereoisomers thereof.

In some embodiments R1 has an average alkyl chain length of from about 8 to about 18, from about 10 to about 14, from about 12 to about 14, or from about 12 to about 13 carbon atoms. In embodiments R1 is a straight chain alkyl. In embodiments R1 is a straight chain alkyl that is bio-based.

In some embodiments M is an alkali metal, an alkaline earth metal, ammonium, alkylammonium, or a mixture thereof. In embodiments M is an alkali metal, such as sodium.

The surfactants can be derived from glycerol (propane-1,2,3-triol), which is a hydrolysis product of fat saponification. Such alkyl glycerol acetal al sulfate and sulfonate anionic surfactants can be produced as described in Piasecki, A., et al; “Synthesis and Surface Properties of Chemodegradable Anionic Surfactants: Diastereomeric (2-n-alkyl-1,3-dioxan-5-yl) sulfates with Monovalent Counter Ions”, J. Surfactants and Detergents, 2000, vol 3(1), pp 59-65 or in PL 175563B1, Example 1. In addition, the sulfonate variants can be found in Jingxi Huagong, 2009, vol 26(4), pp327-330, and Riyong Huaxuepin Kexue, 2010, vol 33(8), p28-31.

The alkyl glycerol acetal sulfate/sulfonate anionic surfactants of Formula I can comprise one of four isomers, or a blend of two diastereomers. The five-membered ring alkyl glycerol acetal sulfate or sulfonate anionic surfactant has an alkyl chain R1 bound both above and below the plane of the five-membered ring relative to group B to provide a pair of diastereomers. In addition, this relative special arrangement also occurs in a six-membered glycerol acetal sulfate and sulfonate ring formed in the same reaction process, giving an additional pair of diastereomers; i.e. four compounds in total, during ring formation.

The alkyl glycerol acetal sulfate/sulfonate anionic surfactant can have a weight average degree of branching of less than 30%, preferably less than 20%, more preferably less than 10%, and most preferably the alkyl chain of the glycerol acetal sulfate/sulfonate anionic surfactant is linear.

The weight average degree of branching for an anionic surfactant mixture of Formula 1 can be calculated using the following formula:

Weight average degree of branching (%)=[(x1* wt % branched aldehyde 1 in aldehyde 1+x2*wt % branched aldehyde 2 in aldehyde 2+ . . . )/(x1+x2+ . . . )]*100

wherein x1, x2, . . . are the weight in grams of each aldehyde in the total mixture of the aldehydes which were used as starting material before acetal formation and subsequent sulfation/sulfonation to produce the alkyl glycerol acetal sulfate or sulfonate anionic surfactant. In the weight average degree of branching calculation, the weight of each alkyl (branched and unbranched) aldehyde used to form the alkyl sulfate anionic surfactant is used.

The anionic surfactant can comprise both the alkyl glycerol acetal sulfate or sulfonate and the corresponding alkyl sulfate anionic surfactant in a weight ratio of from about 1:9 to about 2:1 about 1:5 to about 1:1, or from about 1:4 to about 1:2. Without wishing to be bound by theory, it is believed that a mixture provides a surfactant packing which balances performance, low temperature stability and robustness against water hardness variations.

Suitable examples of commercially available alkyl sulfate anionic surfactants include, those derived from alcohols sold under the Neodol® brand-name by Shell Oil Company, The Hague, Netherlands, or the Lial®, Isalchem®, and Safol® brand-names by Sasol, Sandton, South Africa, and Exxal® brand-name by Exxon-Mobil, Irving, Tex., and Lutensol® brand-name by BASF, Ludwigshafen, Germany, or some of the natural alcohols produced by The Procter & Gamble Company, Cincinnati, Ohio.

The surfactant system may comprise further anionic surfactant, including p-alkylbenzene sulfonic acid (HLAS), paraffin sulfonates, olefin sulfonates, methyl ester sulfonates, isethionates, glutamates, sarcosinates, glycinates, taurates, ether carboxylates, sophorolipids, rhamnolipids, or sulfosuccinate anionic surfactants.

The detergent compositions may comprise one or more additional surfactants (e.g., a third surfactant, a fourth surfactant), such as anionic surfactants, nonionic surfactants, cationic surfactants, zwitterionic surfactants, amphoteric surfactants, ampholytic surfactants, and mixtures thereof. The additional surfactant may be a detersive surfactant, which those of ordinary skill in the art will understand to encompass any surfactant or mixture of surfactants that provide sudsing, cleaning, stain removing, or laundering benefit to soiled material.

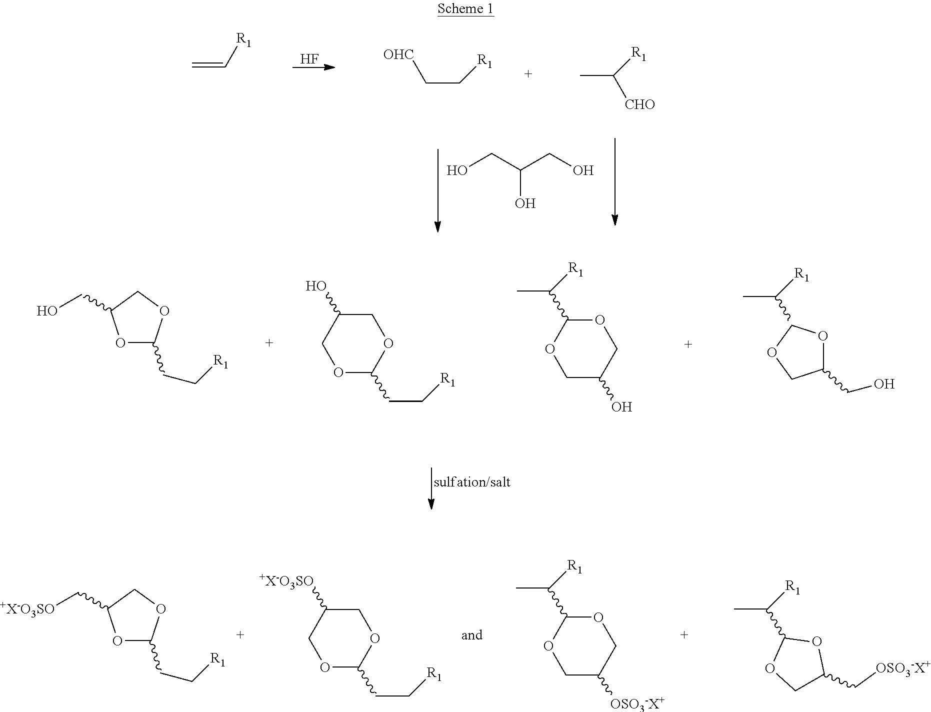

Embodiments of the invention involve the hydroformylation (HF) process of terminal alkenes (Scheme 1) to provide aldehyde mixtures and as exemplified by Lui, C.; et al, Industrial and Engineering Chemistry Research, 2019, 58(47), p. 21295., followed by acetal formation with glycerol to provide a mixture of 5- and 6-membered ring acetal alcohols as sulfation precursors. These materials are then converted to useful sulfate surfactants using procedures as outlined in Al-Horani, R. A.; et al; “Chemical Sulfation of Small Molecules- Advances and Challenges” Tetranderon, 2010, 66(16), p. 2907-2918.

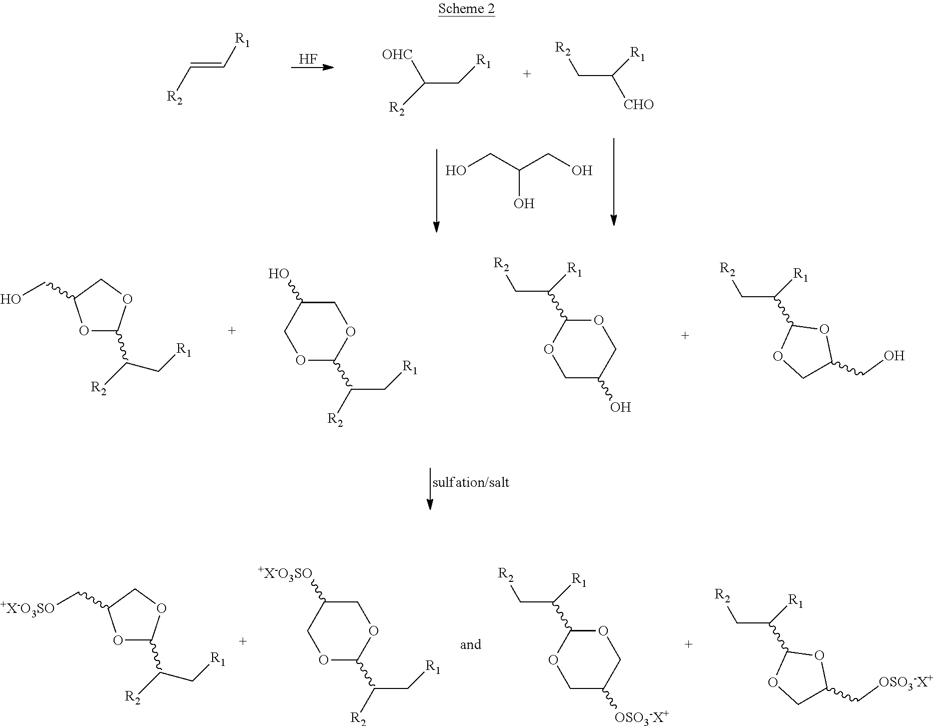

Embodiments of the invention involve the hydroformylation (HF) process of internal alkenes (Scheme 2) as exemplified by Shirakawa,S.; et al; Journal of New Chemistry, 2001, 25(6), p. 777-779. , followed by acetal formation with glycerol to provide a mixture of five- and six-membered ring acetals as sulfation precursors. These materials are then converted to useful sulfate surfactants using procedures as outlined in Al-Horani, R. A.; et al; “Chemical Sulfation of Small Molecules- Advances and Challenges” Tetranderon, 2010, 66(16), p. 2907-2918.

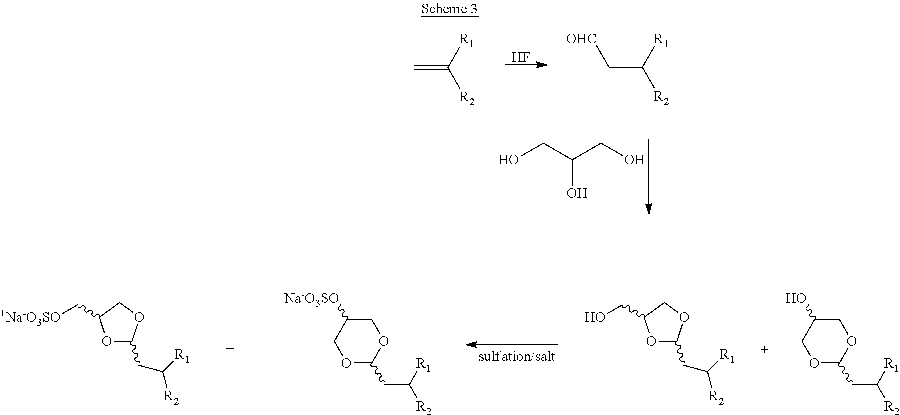

In embodiments of the invention glycerol acetal sulfate surfactants can be produced using the sequence of conversions depicted in Scheme 3 involving the use of petroleum-based or bio-based vinylidene olefin feedstocks. For example, olefin feedstocks of infinite variety can be converted into aldehyde precursors using hydroformylation processes (HF), such as the Oxo Process (Franke, R.; et. al; “Applied Hydroformylation”, Chemical Reviews, 2000, 112(11), p. 56′75-5′732, or Breit, B.; et al; Chemistry. A European Journal, 2001, 7(14), p. 3106-3121. The aldehyde precursors are then treated with glycerol under various conditions to provide a mixture of 5- and 6-membered ring acetals as sulfation precursors. These precursors are then converted to useful sulfate surfactants using procedures as outlined in Al-Horani, R. A.; et al; “Chemical Sulfation of Small Molecules- Advances and Challenges” Tetranderon, 2010, 66(16), p. 2907-2918.

In embodiments, the bio-based content of the glycerol acetal sulfate surfactants is greater than about 3%. In another embodiment, the bio-based content of the glycerol acetal sulfate surfactants is greater than about 12%. In another embodiment, the bio -based content of the glycerol acetal sulfate surfactants is greater than about 30%. In yet another embodiment, the bio-based content of the glycerol acetal sulfate surfactants is greater than about about 90%.

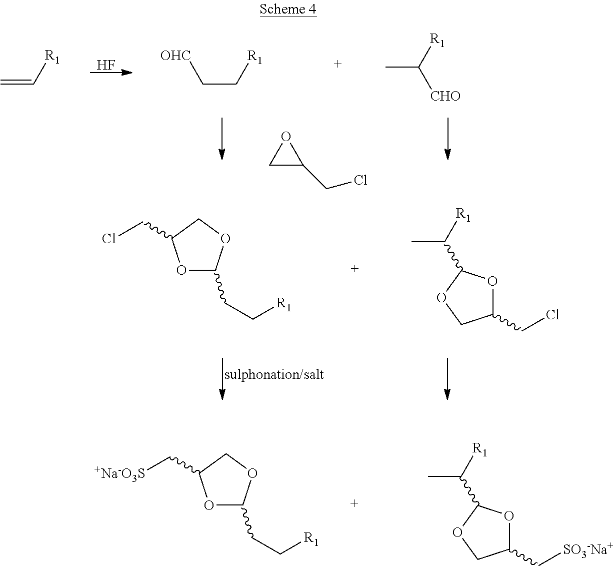

In embodiments of the invention glycerol acetal sulfonate surfactants can be produced using the conversions depicted in Scheme 4 using epichlorohydrin to provide a chlorinated acetal as a single five-membered ring isomer. This chlorinated acetal is then converted to a sulfonate surfactant as described by Zhai, W.; et al: “Synthesis of new acetal anionic surfactants.” Riyong Huaxuepin Kexue, 2010, 33 (8), p. 28-31.

Aldehydes used in the conversions depicted in Schemes 1-4 can be derived from carboxylic acids enzymatically via biochemical processes such as the route depicted in Scheme 5 as described by Kaehne,F. et. al; “A recombinant α-dioxygenase from rice to produce fatty aldehydes using E. coli.”, Applied Microbiol. Biotechnol., 2011, 90 (3), p. 989-995.

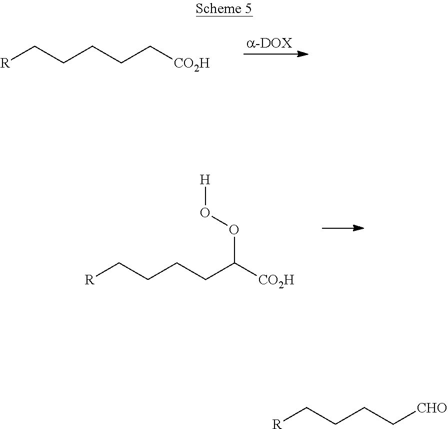

Preferred aldehydes for conversion to the acetals may be petroleum-based and/or bio-based. Preferred aldehydes include, but are not limited to: oleyl aldehyde, dodecanal, decanal, nonanal, octanal, heptanal, hexanal, geranial, 2-ethyl hexanal, 2-propylheptanal, tridecanal, 2-methyldodecanal, 2-ethylundecanal, 2-propyldecanal, 2-butylnonanal, 2-pentyloctanal, (Z)-2-benzylidene hexenal, citral, anisic aldehyde, florhydral, melonal, helional, and isotridecanals including branched tridecanals made using OXO processes with branched alpha olefins derived from propylene, butylene, ethylene and mixtures thereof.

The glycerol acetal surfactants in TABLE 1 have been synthesized.

TABLE 1

| Compound | Name |
|---|---|
| 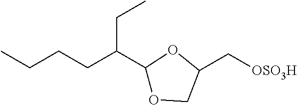 | (2-(heptan-3-yl)-1,3-dioxolan-4-yl)methyl hydrogen sulfate |
| 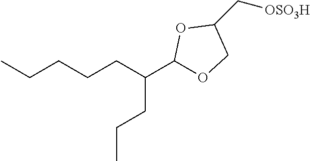 | (2-(nonan-4-yl)-1,3-dioxolan-4-yl) methyl hydrogen sulfate |
| 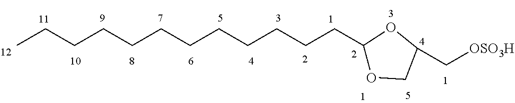 | (2-dodecyl-1,3-dioxolan-4-yl) methyl hydrogen sulfate |
| 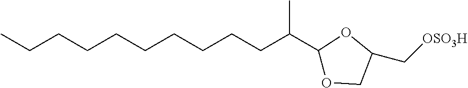 | (2-(dodecan-2-yl)-1,3-dioxolan-4-yl) methyl hydrogen sulfate |
| 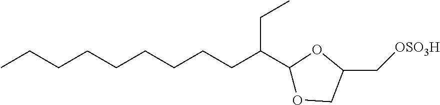 | (2-(dodecan-3-yl)-1,3-dioxolan-4-yl) methyl hydrogen sulfate |
| 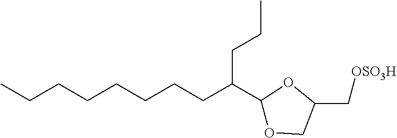 | (2-(dodecan-4-yl)-1,3-dioxolan-4-yl) methyl hydrogen sulfate |
| 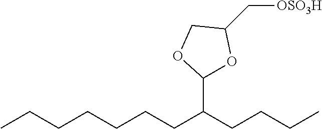 | (2-(dodecan-5-yl)-1,3-dioxolan-4-yl) methyl hydrogen sulfate |
| 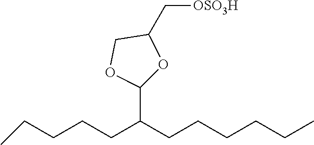 | (2-(dodecan-6-yl)-1,3-dioxolan-4-yl) methyl hydrogen sulfate |
| 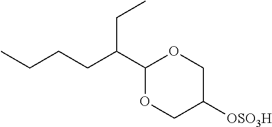 | 2-(heptan-3-yl)-1,3-dioxan-5-yl hydrogen sulfate |
| 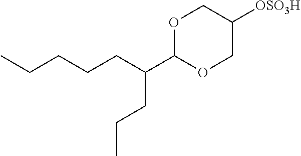 | 2-(nonan-4-yl)-1,3-dioxan-5-yl hydrogen sulfate |
| 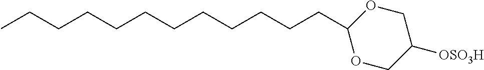 | 2-dodecyl-1,3-dioxan-5-yl hydrogen sulfate |
| 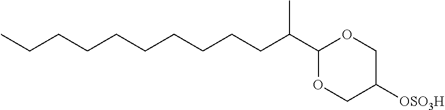 | 2-(dodecan-2-yl)-1,3-dioxan-5-yl hydrogen sulfate |
| 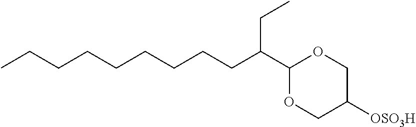 | 2-(dodecan-3-yl)-1,3-dioxan-5-yl hydrogen sulfate |
| 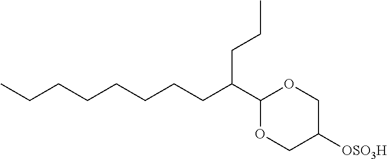 | 2-(dodecan-4-yl)-1,3-dioxan-5-yl hydrogen sulfate |
| 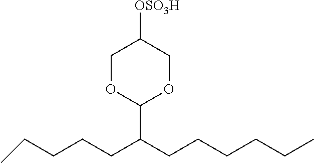 | 2-(dodecan-6-yl)-1,3-dioxan-5-yl hydrogen sulfate |
| 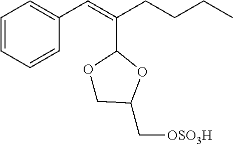 | (Z)-(2-(1-phenylhex-1-en-2-yl)-1,3-dioxolan-4- methyl hydrogen sulfate |
| 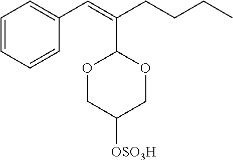 | (Z)-2-(1-phenylhex-1-en-2-yl)-1,3-dioxan-5 hydrogen sulfate |
| 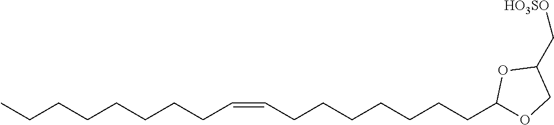 | (Z)-(2-(heptadec-8-en-1-yl)-1,3-dioxolan-4-yl) methyl hydrogen sulfate |
| 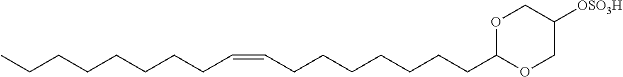 | (Z)-2-(heptadec-8-en-1-yl)-1,3-dioxan-5-yl hydrogen sulfate |
| 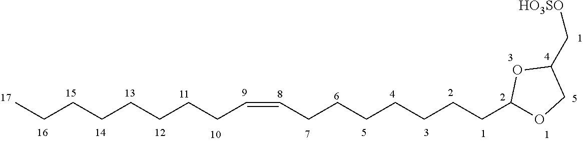 | (Z)-(2-(heptadec-8-en-1-yl)-1,3- dioxolan-4-yl)methanesulfonic acid |
| 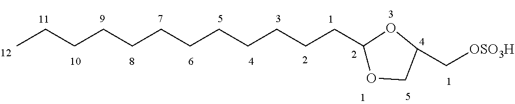 | (2-dodecyl-1,3-dioxolan-4-yl) methyl hydrogen sulfate |
| 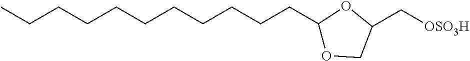 | (2-undecyl-1,3-dioxolan-4-yl) methyl hydrogen sulfate |
| 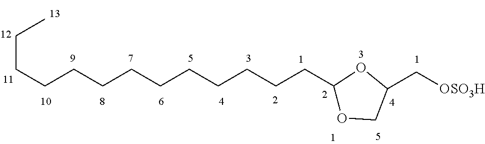 | (2-tridecyl-1,3-dioxolan-4-yl) methyl hydrogen sulfate |
| 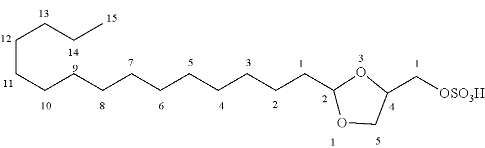 | (2-pentadecyl-1,3-dioxolan-4-yl) methyl hydrogen sulfate |
| 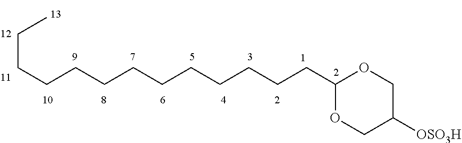 | 2-tridecyl-1,3-dioxan-5-yl hydrogen sulfate |
| 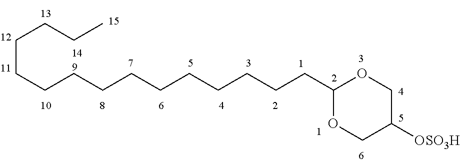 | 2-pentadecyl-1,3-dioxan-5-yl hydrogen sulfate |
| 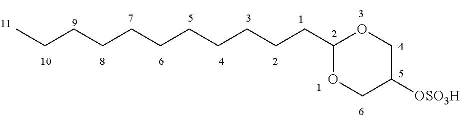 | 2-undecyl-1,3-dioxan-5-yl hydrogen sulfate |
| 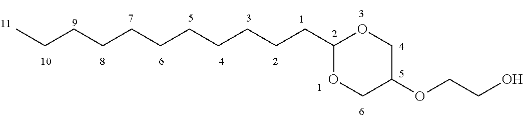 [SMILES: CCCCCCCCCCCC1OCC(OCCO)CO1] | 2-((2-undecyl-1,3-dioxan-5-yl) oxy)ethan-1-ol |
| 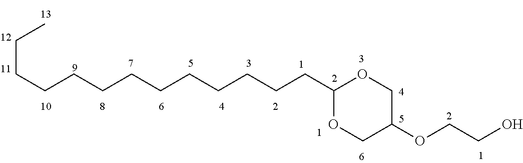 [SMILES: CCCCCCCCCCCCCC1OCC(OCCO)CO1] | 2-((2-tridecyl-1,3-dioxan-5-yl) oxy)ethan-1-ol |
| 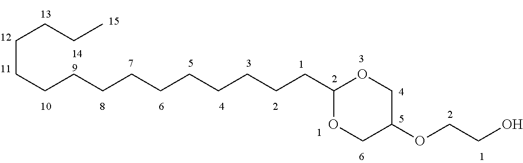 [SMILES: CCCCCCCCCCCCCCCC1OCC(OCCO)CO1] | 2-((2-pentadecyl-1,3-dioxan-5-yl) oxy)ethan-1-ol |
| 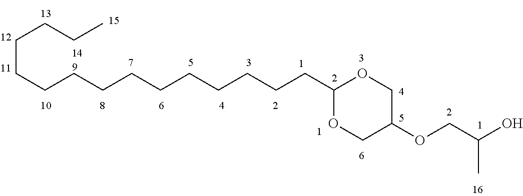 [SMILES: CCCCCCCCCCCCCCCC1OCC(OCC(C)O)CO1] | 1-((2-pentadecyl-1,3-dioxan-5-yl) oxy)propan-2-ol |
| 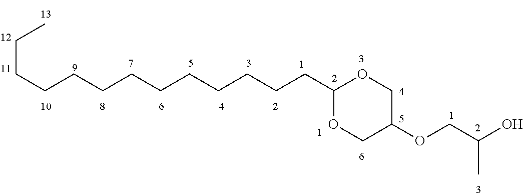 [SMILES: CCCCCCCCCCCCCC1OCC(OCC(C)O)CO1] | 1-((2-tridecyl-1,3-dioxan-5-yl) oxy)propan-2-ol |
| 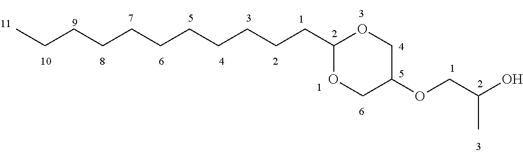 [SMILES: CCCCCCCCCCCC1OCC(OCC(C)O)CO1] | 1-((2-undecyl-1,3-dioxan-5-yl) oxy)propan-2-ol |
| 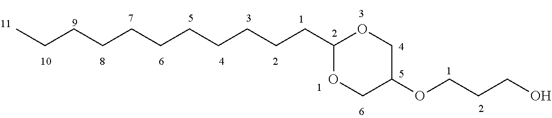 [SMILES: CCCCCCCCCCCC1OCC(OCCCO)CO1] | 3-((2-undecyl-1,3-dioxan-5-yl) oxy)propan-1-ol |
| 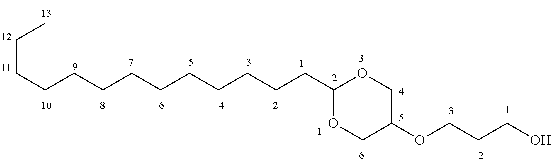 [SMILES: CCCCCCCCCCCCCC1OCC(OCCCO)CO1] | 3-((2-tridecyl-1,3-dioxan-5-yl) oxy)propan-1-ol |
| 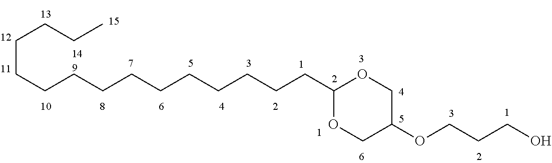 [SMILES: CCCCCCCCCCCCCCCC1OCC(OCCCO)CO1] | 3-((2-pentadecyl-1,3-dioxan-5-yl) oxy)propan-1-ol |
| 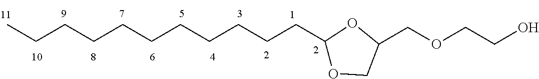 [SMILES: CCCCCCCCCCCC1OCC(COCCO)O1] | 2-((2-undecyl-1,3-dioxolan-4-yl) methoxy)ethan-1-ol |
| 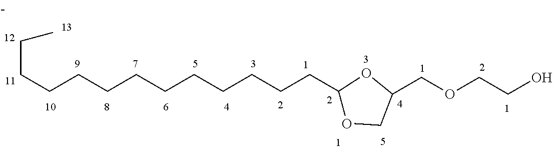 [SMILES: CCCCCCCCCCCCCC1OCC(COCCO)O1] | 2-((2-tridecyl-1,3-dioxolan-4-yl) methoxy)ethan-1-ol |
| 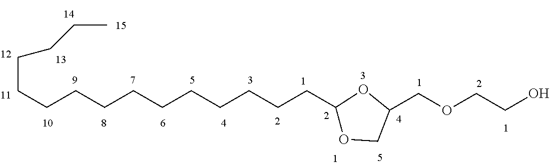 [SMILES: CCCCCCCCCCCCCCCC1OCC(COCCO)O1] | 2-((2-pentadecyl-1,3-dioxolan-4-yl) methoxy)ethan-1-ol |
| 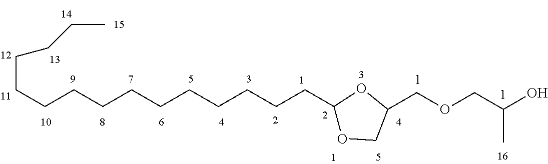 [SMILES: CCCCCCCCCCCCCCCC1OCC(COCC(C)O)O1] | 1-((2-pentadecyl-1,3-dioxolan-4-yl) methoxy)propan-2-ol |
| 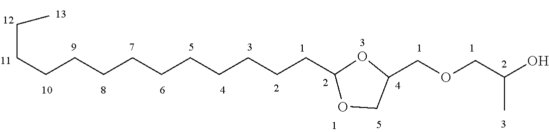 [SMILES: CCCCCCCCCCCCCC1OCC(COCC(C)O)O1] | 1-((2-tridecyl-1,3-dioxolan-4-yl) methoxy)propan-2-ol |
| 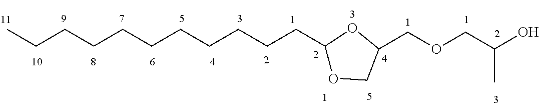 [SMILES: CCCCCCCCCCCC1OCC(COCC(C)O)O1] | 1-((2-undecyl-1,3-dioxolan-4-yl) methoxy)propan-2-ol |
| 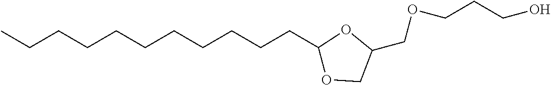 [SMILES: CCCCCCCCCCCC1OCC(COCCCO)O1] | 3-((2-undecyl-1,3-dioxolan-4-yl) methoxy)propan-1-ol |
| 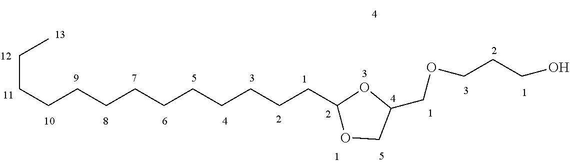 [SMILES: CCCCCCCCCCCCCC1OCC(COCCCO)O1] | 3-((2-tridecyl-1,3-dioxolan-4-yl) methoxy)propan-1-ol |
| 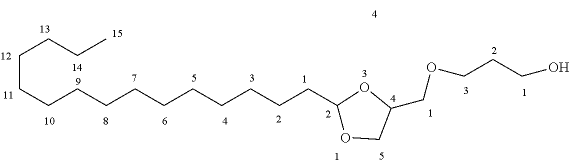 [SMILES: CCCCCCCCCCCCCCCC1OCC(COCCCO)O1] | 3-((2-pentadecyl-1,3-dioxolan-4-yl) methoxy)propan-1-ol |

Liquid Detergent Composition

A detergent composition, which includes a GAS of the present invention, may be a hand dishwashing detergent composition in liquid form. The liquid detergent composition is preferably an aqueous detergent composition. As such, the composition can comprise from 50% to 85%, preferably from 50% to 75%, by weight of the total composition of water.

The pH of the composition may be at least 7.0, preferably from about 7.0 to about 12.0, or more preferably from about 7.5 to about 10.0, as measured at 10% dilution in distilled water at 20° C. The pH of the composition can be adjusted using pH modifying ingredients known in the art.

The composition can be Newtonian or non-Newtonian, with certain embodiments being Newtonian. The composition may have a viscosity of from about 10 mPa·s to about 10,000 mPa·s, from about 100 mPa·s to about 5,000 mPa·s, from about 300 mPa·s to about 2,000 mPa·s, or from about 500 mPa·s to about 1,500 mPa·s, alternatively combinations thereof. The viscosity is measured with a Brookfield RT Viscometer using spindle 21 at 20 RPM at 25° C.

Surfactant System

A detergent composition can comprise from about 5% to about 50%, from about 8% to about 45%, or from about 15% to about 40%, by weight of the total composition of a surfactant system. The surfactant system comprises anionic surfactant and a co-surfactant which is selected from the group consisting of an amphoteric surfactant, a zwitterionic surfactant and mixtures thereof. In order to improve surfactant packing after dilution and hence improve robustness against hardness variations and suds mileage, the surfactant system can comprise the anionic surfactant and co-surfactant in a weight ratio of co-surfactant: anionic surfactant from about 1:1 to about 1:9, about 1:2 to about 1:5, or about 1:2.5 to about 1:4.

Anionic Surfactant:

A detergent composition may comprise from about 60% to about 90%, from about 65% to about 85%, or from about 70% to about 80% by weight of the surfactant system of the anionic surfactant. The anionic surfactant can comprise from about 10% to about 100%, from about 20% to about 80%, or from about 25% to about 35% by weight of the anionic surfactant of a glycerol acetal sulfate or sulfonate anionic surfactant.

Co-Surfactant:

A detergent composition may further comprise a co-surfactant selected from the group consisting of an amphoteric surfactant, a zwitterionic surfactant and mixtures thereof, as part of the surfactant system. The composition may comprise from about 0.1% to about 20%, from about 0.5% to about 15%, or from about 2% to about 10% by weight of the detergent composition of the co-surfactant.

The surfactant system of the detergent composition may comprise from about 10% to about 40%, from about 15% to about 35%, or from about 20% to about 30%, by weight of the surfactant system of a co-surfactant.

The co-surfactant may be at least one of an amphoteric surfactant, a zwitterionic surfactant, and mixtures thereof. In embodiments the co-surfactant may be an amphoteric surfactant, such as an amine oxide surfactant.

The amine oxide surfactant can be linear or branched, with certain embodiments being linear. Suitable linear amine oxides are typically water-soluble, and characterized by the formula R1-N(R2)(R3) 0 wherein R1 is a C8-18 alkyl, and the R2 and R3 moieties are selected from the group consisting of C1-3 alkyl groups, C1-3 hydroxyalkyl groups, and mixtures thereof. For instance, R2 and R3 can be selected from the group consisting of: methyl, ethyl, propyl, isopropyl, 2-hydroxethyl, 2-hydroxypropyl and 3-hydroxypropyl, and mixtures thereof, wherein in certain embodiments methyl may be one or both of R2 and R3. The linear amine oxide surfactants in particular may include linear C10-C18 alkyl dimethyl amine oxides and linear C10-C18 dihydroxyethyl amine oxides.

The amine oxide surfactant may be at least one of: alkyl dimethyl amine oxide, alkyl amido propyl dimethyl amine oxide, and mixtures thereof. In certain embodiments alkyl dimethyl amine oxides may be used, such as C8-18 alkyl dimethyl amine oxides, or C10-16 alkyl dimethyl amine oxides (such as coco dimethyl amine oxide). Suitable alkyl dimethyl amine oxides include C10 alkyl dimethyl amine oxide surfactant, C10-12 alkyl dimethyl amine oxide surfactant, C12-C14 alkyl dimethyl amine oxide surfactant, and mixtures thereof. In certain embodiments the amine oxide surfactant may be C12-C14 alkyl dimethyl amine oxide and the alkyl chain of the alkyl dimethyl amine oxide may be a linear alkyl chain, such as a C12-C14 alkyl chain, for example a C12-C14 alkyl chain derived from coconut oil, palm kernel oil, or from Ziegler alcohols.

Alternative suitable amine oxide surfactants include mid-branched amine oxide surfactants. As used herein, “mid-branched” means that the amine oxide has one alkyl moiety having n1 carbon atoms with one alkyl branch on the alkyl moiety having n2 carbon atoms. The alkyl branch is located on the a carbon from the nitrogen on the alkyl moiety. This type of branching for the amine oxide is also known in the art as an internal amine oxide. The total sum of n1 and n2 can be from about 10 to about 24 carbon atoms, from about 12 to about 20, or from about 10 to about 16. The number of carbon atoms for the one alkyl moiety (n1) may be the same or similar to the number of carbon atoms as the one alkyl branch (n2) such that the one alkyl moiety and the one alkyl branch are symmetric. As used herein “symmetric” means that |n1-n2| in certain embodiments is less than or equal to about 5, less than or equal to about 4, or from 0 to about 4 carbon atoms in at least 50 wt % or at least 75 wt % to 100 wt % of the mid-branched amine oxides for use herein. The amine oxide further comprises two moieties, independently selected from a C1-3 alkyl, a C1-3 hydroxyalkyl group, or a polyethylene oxide group containing an average of from about 1 to about 3 ethylene oxide groups. The two moieties may be selected from a C1-3 alkyl, more preferably both are selected as C1 alkyl.

Alternatively, the amine oxide surfactant can be a mixture of amine oxides comprising a mixture of low-cut amine oxide and mid-cut amine oxide. The amine oxide can then comprise:
a) from about 10% to about 45% by weight of the amine oxide of low-cut amine oxide of formula R1R2R3AO wherein R1 and R2 are independently selected from hydrogen, C1-C4 alkyls or mixtures thereof, and R3 is selected from C10 alkyls and mixtures thereof; and
b) from about 55% to about 90% by weight of the amine oxide of mid-cut amine oxide of formula R4R5R6AO wherein R4 and R5 are independently selected from hydrogen, C1-C4 alkyls or mixtures thereof, and R6 is selected from C12-C16 alkyls or mixtures thereof

In embodiments a low-cut amine oxide for use herein R3 is n-decyl, wherein both R1 and R2 may be methyl. In the mid-cut amine oxide of formula R4R5R6AO, R4 and R5 may be both methyl.

In embodiments the amine oxide comprises less than about 5% or less than about 3%, by weight of the amine oxide of an amine oxide of formula R7R8R9AO wherein R7 and R8 are selected from hydrogen, C1-C4 alkyls and mixtures thereof and wherein R9 is selected from C8 alkyls and mixtures thereof. Limiting the amount of amine oxides of formula R7R8R9AO improves both physical stability and suds mileage.

Suitable zwitterionic surfactants include betaine surfactants. Such betaine surfactants include alkyl betaines, alkyl N,N-dimethyl betaines, alkylamidobetaine, amidazoliniumbetaine, alkyl sulfobetaines (INCI Sultaines) as well as the alkyl phosphobetaines, and preferably meets formula (I):

R¹—[CO—X(CH₂)ₙ]ₓ—N⁺(R²)(R₃)-(CH₂)ₘ-[CH(OH)-CH₂]_{y}—Y⁻

wherein in formula (I),

R1 is at least one of: a saturated or unsaturated C6-22 alkyl residue, preferably C8-18 alkyl residue, a saturated C10-16 alkyl residue, or a saturated C12-14 alkyl residue;

X is selected from the group consisting of: NH, NR4 wherein R4 is a C1-4 alkyl residue, O, and S; n is an integer from 1 to 10, preferably 2 to 5, more preferably 3′

x is 0 or 1;

R2 and R3 are independently at least one of: a C1-4 alkyl residue, hydroxy substituted such as a hydroxyethyl, and mixtures thereof, preferably both R2 and R3 are methyl;

m is an integer from 1 to 4;

y is 0 or 1; and

Y is at least one of: COO, SO3, OPO(OR5O or P(O)(OR5)O, wherein R5 is H or a C1-4 alkyl residue.

Betaines may be the alkyl betaines of formula (IIa), the alkyl amido propyl betaine of formula (IIb), the sulfobetaines of formula (IIc) and the amido sulfobetaine of formula (IId):

R¹—N⁺(CH₃)₂—CH₂COO⁻ (IIa)

R¹—CO—NH—(CH₂)₃—N⁺(CH₃)₂—CH₂COO⁻ (IIb)

R¹—N⁺(CH₃)₂—CH₂CH(OH)CH₂SO₃⁻ (IIc)

R¹—CO—NH—(CH₂)₃—N⁺(CH₃)₂—CH₂CH(OH)CH₂SO₃⁻ (IId)

in which R1 has the same meaning as in formula (I). In embodiments the carbobetaines [i.e. wherein Y′=COO— in formula (I)] of formulae (IIa) and (IIb), may be used, such as the alkylamidobetaine of formula (IIb).

Suitable betaines can be selected from the group consisting or [designated in accordance with INCI]: capryl/capramidopropyl betaine, cetyl betaine, cetyl amidopropyl betaine, cocamidoethyl betaine, cocamidopropyl betaine, cocobetaines, decyl betaine, decyl amidopropyl betaine, hydrogenated tallow betaine/amidopropyl betaine, isostearamidopropyl betaine, lauramidopropyl betaine, lauryl betaine, myristyl amidopropyl betaine, myristyl betaine, oleamidopropyl betaine, oleyl betaine, palmamidopropyl betaine, palmitamidopropyl betaine, palm-kernelamidopropyl betaine, stearamidopropyl betaine, stearyl betaine, tallowamidopropyl betaine, tallow betaine, undecylenamidopropyl betaine, undecyl betaine, and mixtures thereof. In embodiments betaines may be at least one of: cocamidopropyl betaine, cocobetaines, lauramidopropyl betaine, lauryl betaine, myristyl amidopropyl betaine, myristyl betaine, and mixtures thereof. In embodiments cocamidopropyl betaine may be used.

Nonionic Surfactant:

The surfactant system can further comprise a nonionic surfactant, In embodiments a nonionic surfactant may be at least one of: alkyl alkoxylated nonionic surfactants, alkylpolyglucosides, and mixtures thereof. In embodiments the nonionic surfactant may be alkylpolyglucosides.

The surfactant system can comprise from about 1% to about 25%, preferably from about 1.25% to about 20%, more preferably from about 1.5% to about 15%, most preferably from about 1.5% to about 5%, by weight of the surfactant system, of the non-ionic surfactant.

The alkoxylated non-ionic surfactant can be a linear or branched, primary or secondary alkyl alkoxylated non-ionic surfactant, such as an alkyl ethoxylated non-ionic surfactant, which may comprise on average from about 9 to about 15 carbon atoms in its alkyl chain, or from about 10 to about 14 carbon atoms, and on average from about 5 to about 12 units of ethylene oxide per mole of alcohol, from about 6 to about 10 units, or from about 7 to about 8 units.

A detergent composition can comprise alkyl polyglucoside (“APG”) surfactant. The addition of alkyl polyglucoside surfactants have been found to improve sudsing beyond that of comparative nonionic surfactants such as alkyl ethoxylated surfactants.

The alkyl polyglucoside surfactant may be a C8-C16 alkyl polyglucoside surfactant, such as a C8-C14 alkyl polyglucoside surfactant. The alkyl polygluco side may have an average degree of polymerization of between about 0.1 to about 3, between about 0.5 to about 2.5, or between about 1 to about 2. In embodiments the alkyl polyglucoside surfactant may have an average alkyl carbon chain length between about 10 to about 16, between about 10 to about 14, or between about 12 to about 14, with an average degree of polymerization of between about 0.5 to about 2.5 between about 1 to about 2, or between about 1.2 to about 1.6.

C8-C16 alkyl polyglucosides are commercially available from several suppliers (e.g., Simusol® surfactants from Seppic Corporation, Paris, France; and Glucopon® 600 CSUP, Glucopon® 650 EC, Glucopon® 600 CSUP/MB, and Glucopon® 650 EC/MB, from BASF Corporation, Ludwigshafen, Germany).

Amphiphilic Alkoxylated Polyalkyleneimine:

A detergent composition may further comprise from about 0.05% to about 2%, preferably from about 0.07% to about 1% by weight of the total composition of an amphiphilic polymer. Suitable amphiphilic polymers can be at least one of: amphiphilic alkoxylated polyalkyleneimine and mixtures thereof. The amphiphilic alkoxylated polyalkyleneimine polymer has been found to reduce gel formation on the hard surfaces to be cleaned when the liquid composition is added directly to a cleaning implement (such as a sponge) before cleaning and consequently brought in contact with heavily greased surfaces, especially when the cleaning implement comprises a low amount to nil water such as when light pre-wetted sponges are used.

The amphiphilic alkoxylated polyalkyleneimine may be an alkoxylated polyethyleneimine polymer comprising a polyethyleneimine backbone having a weight average molecular weight range of from about 100 to about 5,000, from about 400 to about or 2,000, more preferably from about 400 to about 1,000 Daltons. The polyethyleneimine backbone comprises the following modifications:
(i) one or two alkoxylation modifications per nitrogen atom, dependent on whether the modification occurs at an internal nitrogen atom or at an terminal nitrogen atom, in the polyethyleneimine backbone, the alkoxylation modification consisting of the replacement of a hydrogen atom on by a polyalkoxylene chain having an average of about 1 to about 50 alkoxy moieties per modification, wherein the terminal alkoxy moiety of the alkoxylation modification is capped with hydrogen, a C1-C4 alkyl or mixtures thereof;
(ii) a substitution of one C1-C4 alkyl moiety and one or two alkoxylation modifications per nitrogen atom, dependent on whether the substitution occurs at an internal nitrogen atom or at a terminal nitrogen atom, in the polyethyleneimine backbone, the alkoxylation modification consisting of the replacement of a hydrogen atom by a polyalkoxylene chain having an average of about 1 to about 50 alkoxy moieties per modification wherein the terminal alkoxy moiety is capped with hydrogen, a C 1-C4 alkyl or mixtures thereof; or
(iii) a combination thereof.

For example, but not limited to, shown below are possible modifications to terminal nitrogen atoms in the polyethyleneimine backbone where R represents an ethylene spacer and E represents a C1-C4 alkyl moiety and X— represents a suitable water soluble counterion:

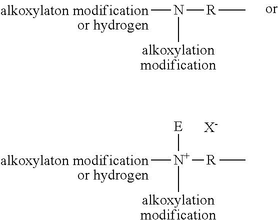

Also, for example, but not limited to, shown below are possible modifications to internal nitrogen atoms in the polyethyleneimine backbone where R represents an ethylene spacer and E represents a C₁-C4 alkyl moiety and X— represents a suitable water soluble counterion:

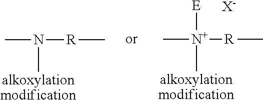

The alkoxylation modification of the polyethyleneimine backbone consists of the replacement of a hydrogen atom by a polyalkoxylene chain having an average of about 1 to about 50 alkoxy moieties, from about 20 to about 45 alkoxy moieties, or from about 30 to about 45 alkoxy moieties. The alkoxy moieties may be from ethoxy (EO), propoxy (PO), butoxy (BO), and mixtures thereof. The polyalkoxylene chain may be selected from ethoxy/propoxy block moieties. In embodiments, the polyalkoxylene chain is ethoxy/propoxy block moieties having an average degree of ethoxylation from about 3 to about 30 and an average degree of propoxylation from about 1 to about 20, or ethoxy/propoxy block moieties having an average degree of ethoxylation from about 20 to about 30 and an average degree of propoxylation from about 10 to about 20.

In embodiments the ethoxy/propoxy block moieties have a relative ethoxy to propoxy unit ratio between about 3 to about 1 and about 1 to 1, in certain embodiments between about 2 to about 1 and about 1 to about 1.

The modification may result in permanent quaternization of the polyethyleneimine backbone nitrogen atoms. The degree of permanent quaternization may be from 0% to about 30% of the polyethyleneimine backbone nitrogen atoms. In embodiments there may be less than 30% of the polyethyleneimine backbone nitrogen atoms permanently quaternized. In embodiments the degree of quaternization is about 0%.

An amphiphilic alkoxylated polyethyleneimine polymer may have the general structure of formula (II):

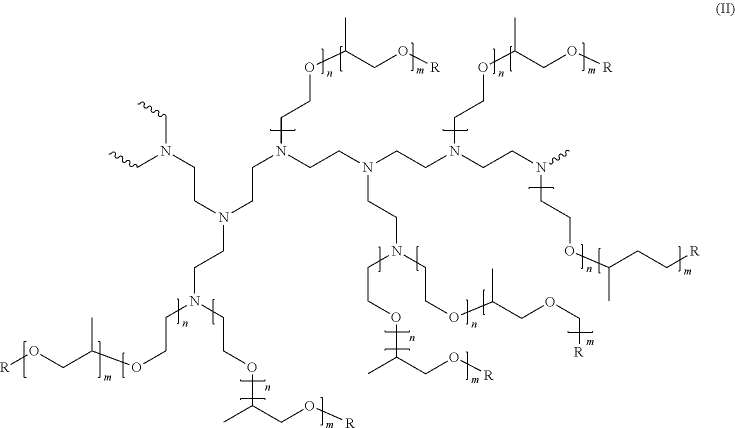

wherein the polyethyleneimine backbone has a weight average molecular weight of about 600, n of formula (II) has an average of about 10 meaning the oligomeric blocks of ethylene oxide have about 10 units of ethylene oxide on average m of formula (II) has an average of about 7 meaning the oligomeric blocks of propylene oxide have about 7 units of propylene oxide on average, and R of formula (II) is at least one of hydrogen, a C₁-C₄ alkyl and mixtures thereof, in certain embodiments hydrogen. The degree of permanent quaternization of formula (II) may be from 0% to about 22% of the polyethyleneimine backbone nitrogen atoms. The molecular weight of this amphiphilic alkoxylated polyethyleneimine polymer preferably is between 10,000 and 15,000 Da.

In embodiments, the amphiphilic alkoxylated polyethyleneimine polymer has the general structure of formula (II) but wherein the polyethyleneimine backbone has a weight average molecular weight of about 600 Da, n of Formula (II) has an average of about 24 meaning the oligomeric blocks of ethylene oxide have about 24 units of ethylene oxide on average, m of Formula (II) has an average of about 16 meaning the oligomeric blocks of propylene oxide have about 16 units of propylene oxide on average, and R of Formula (II) is at least one of hydrogen, a C₁-C₄ alkyl and mixtures thereof, in certain embodiments hydrogen. The degree of permanent quaternization of Formula (II) may be from 0% to about 22% of the polyethyleneimine backbone nitrogen atoms, in embodiments it may be 0%. The molecular weight of this amphiphilic alkoxylated polyethyleneimine polymer may be between about 25,000 to about 30,000, in embodiments about 28,000 Da.

The amphiphilic alkoxylated polyethyleneimine polymers can be made by the methods described in more detail in PCT Publication No. WO 2007/135645.

Cyclic Polyamine

A detergent composition can comprise a cyclic polyamine having amine functionalities that helps cleaning. The composition may comprise from about 0.1% to about 3%, from about 0.2% to about 2%, or from about 0.5% to about 1%, by weight of the composition, of the cyclic polyamine.

The amine can be subjected to protonation depending on the pH of the cleaning medium in which it is used. Preferred cyclic polyamines have the following Formula (III):

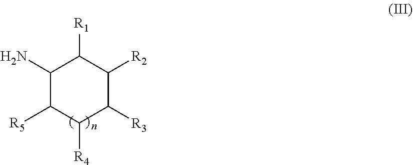

wherein R₁, R₂, R₃, R₄ and Rs are independently at least one of NH2, -H, linear or branched alkyl having from about 1 to about 10 carbon atoms, and linear or branched alkenyl having from about 1 to about 10 carbon atoms, n is from about 1 to about 3, and wherein at least one of the Rs is NH2 and the remaining “Rs” are independently selected from the group consisting of NH2, —H, linear or branched alkyl having about 1 to about 10 carbon atoms, and linear or branched alkenyl having from about 1 to about 10 carbon atoms. The cyclic polyamine may be a diamine, wherein n is 1, R₂ is NH2, and at least one of R₁, R₃, R₄ and Rs is CH3 and the remaining Rs are H.

The cyclic polyamine has at least two primary amine functionalities. The primary amines can be in any position in the cyclic amine but it has been found that in terms of grease cleaning, better performance is obtained when the primary amines are in positions 1,3. It has also been found that cyclic amines in which one of the substituents is -CH3 and the rest are H provided for improved grease cleaning performance.

Accordingly, in embodiments the cyclic polyamine for use with a detergent are cyclic polyamine may be at least one of: 2-methylcyclohexane-1,3-diamine, 4-methylcyclohexane-1,3-diamine and mixtures thereof. These specific cyclic polyamines work to improve suds and grease cleaning profile through-out the dishwashing process when formulated together with the surfactant system of the composition of the present invention.

Additional Ingredients:

A detergent composition may further comprise at least one active comprising: i) a salt, ii) a hydrotrope, iii) an organic solvent, or mixtures thereof.

Salt:

The composition may comprise from about 0.05% to about 2%, from about 0.1% to about 1.5%, or from about 0.5% to about 1%, by weight of the total composition of a salt, in embodiments a monovalent or divalent inorganic salt, or a mixture thereof, for example—sodium chloride, sodium sulfate, or mixtures thereof.

Hydrotrope:

The composition may comprise from about 0.1% to about 10%, from about 0.5% to about 10%, or from about 1% to about 10% by weight of the total composition of a hydrotrope or a mixture thereof, for example sodium cumene sulfonate.

Organic Solvent:

A composition can comprise from about 0.1% to about 10%, from about 0.5% to about 10%, or from about 1% to about 10% by weight of the total composition of an organic solvent. Suitable organic solvents include: alcohols, glycols, glycol ethers, and mixtures thereof, preferably alcohols, glycols, and mixtures thereof. In embodiments ethanol is the organic solvent. In embodiments polyalkyleneglycols, such as polypropyleneglycol, are the organic solvents.

Adjunct Ingredients

A detergent composition may optionally comprise a number of other adjunct ingredients such as builders (preferably citrate), chelants, conditioning polymers, other cleaning polymers, surface modifying polymers, structurants, emollients, humectants, skin rejuvenating actives, enzymes, carboxylic acids, scrubbing particles, perfumes, perfume delivery aids, enzymes, hueing dyes, malodor control agents, pigments, dyes, opacifiers, pearlescent particles, inorganic cations such as alkaline earth metals such as Ca/Mg-ions, antibacterial agents, preservatives, viscosity adjusters (e.g., salt such as NaCl, and other mono-, di- and trivalent salts) and pH adjusters and buffering means (e.g. carboxylic acids such as citric acid, HCl, NaOH, KOH, alkanolamines, carbonates such as sodium carbonates, bicarbonates, sesquicarbonates, and analogs).

### Test Methods

The following assays set forth are used in order that the invention described and claimed herein may be more fully understood.

Suds Generation and Suds Mileage Test Method

The suds generation and suds mileage of test cleaning compositions herein is measured by employing a suds cylinder tester (SCT). The SCT has a set of 8 cylinders. Each cylinder is a Lexan plastic cylinder typically 30 cm long and 8.8 cm internal diameter, with an adhesive ruler affixed to the outside, and a small diameter hole in the top to enable soil additions. Cylinders are together rotated at a rate of 20-22 revolutions per minute (rpm). This method is used to assay the performance of test cleaning compositions to obtain a reading on ability to generate suds as well as the robustness of the suds in the presence of test soil. Approximately 500 ml of the test cleaning solutions are prepared at a surfactant concentration of 359 mg/L in water heated to 60 C and a water hardness of about 257 mg/L made using calcium chloride and magnesium chloride at a 3:1 molar ratio of calcium: magnesium. 300 ml of each test sample solution is poured into a test cylinder in the SCT. When the test solutions have cooled to 45 C, rubber stoppers are put in place to seal the hole in the top of each cylinder.

Rotate cylinders for 2 minutes. Lock in an upright position. Record initial suds height for each cylinder. The height of suds is determined by deducting the height of the liquid layer from the total height of suds and liquid. Continue rotating the cylinders, recording suds height every 2 minutes for a total of 20 minutes. This data represents the Suds Generation of the test cleaning composition. Open the rubber stopper on each cylinder. Add 10.00 g of test soil into each cylinder. Replace the rubber stoppers. Record the starting suds height, and rotate cylinders for 1 minute. Lock in an upright position. Record suds height. Continue rotating the cylinders, recording suds height every 1 minute for a total of 15 minutes. This data represents the Suds Mileage of the test cleaning composition.

Data is recorded as suds generation or suds mileage (cm) vs time (min). Area under the curve (AUC) is calculated using suds generation or suds mileage vs time data and using the trapezoidal rule calculation:

$$\text{?}$$

$$\text{?} \text{indicates text missing or illegible when filed}$$

The AUC results for Suds Generation and Suds Mileage for each test solution are divided by the corresponding AUC result for the relevant test reference and reported as an index (%) compared to the control (100%).

Preparation of the test soil is achieved by standard mixing of the components described below until a homogenous mixture is achieved.

TABLE 2

| Ingredient | Weight % |
|---|---|
| Crisco Oil | 12.730 |
| Crisco shortening | 27.752 |
| Lard | 7.638 |
| Refined Rendered Edible | 51.684 |
| Beef Tallow | |
| Oleic Acid, 90% (Techn) | 0.139 |
| Palmitic Acid, 99+% | 0.036 |
| Stearic Acid, 99+% | 0.021 |

Dynamic Interfacial Tension (DIFT) Test Method

Dynamic Interfacial Tension analysis is performed on a Krüss® DVT30 Drop Volume Tensiometer (Krüs USA, Charlotte, N.C.). The instrument is configured to measure the interfacial tension of an ascending oil drop in aqueous surfactant (surfactant) phase. The test surfactant solutions are prepared at a surfactant concentration of 359 mg/L in water and a water hardness of about 120 mg/L made using calcium chloride and magnesium chloride at a 3:1 molar ratio of calcium: magnesium. The oil used is canola oil (Crisco Pure Canola Oil manufactured by The J.M. Smucker Company). The aqueous surfactant and oil phases are temperature controlled at 22° C. (+/−1° C.), via a recirculating water temperature controller attached to the tensiometer. A dynamic interfacial tension curve is generated by dispensing the oil drops into the aqueous surfactant phase from an ascending capillary with an internal diameter of 0.2540 mm, over a range of flow rates and measuring the interfacial tension at each flow rate. Data is generated at oil dispensing flow rates of 500 uL/min to 1 uL/min with 2 flow rates per decade on a logarithmic scale. Interfacial tension is measured on three oil drops per flow rate and then averaged. Interfacial tension is reported in units of mN/m. Surface age of the oil drops at each flow rate is also recorded and plots may be generated either of interfacial tension (y-axis) versus oil flow rate (x-axis) or interfacial tension (y-axis) versus oil drop surface age (x-axis). Minimum interfacial tension (mN/m) is the lowest interfacial tension at the slowest flow rate, with lower numbers indicating improved performance. Based on instrument reproducibility, differences greater than 0.1 mN/m are significant for interfacial tension values of less than 1 mM/m.

Minimum Surface Tension and Critical Micelle Cohncentration

Surface tension is measured using the Kibron Delta-8 DyneProbe Before every run, the DyneProbes are heated using the Kibron DyneClean furnace. The bottom end of each probe is brought into contact onto a (very) hot surface. Upon contact, the tip of the probe is heated to around 600° C. This ensures consistent and repeatably clean surfaces.

The critical micelle concentration (CMC) of the surfactant is defined by plotting the surface tension of respective surfactant solutions as a function of the logarithm of surfactant concentration at the desired water temperature (20.5 C) and hardness of about 120 mg/L made using calcium chloride and magnesium chloride at a 3:1 molar ratio of calcium: magnesium. The point where the surface versus surfactant concentration slope changes is defined as the CMC value.

A typical CMC determination comprises:

1: Dispensing aliquots of solution: Liquid handling is done using a pipetting robot.

2: Probe cleaning: The DyneProbes are heated to red hot to burn off all contaminants.

3: Immersion and withdrawal: The DyneProbes are positioned above a row of 8 wells and immersed gently. The force on the plate is measured continuously (400 times/s) and upon withdrawal the surface tension is determined.

4: Analysis: Up to 96 data points are acquired in around 3 minutes. These are automatically logged to a PC and the CMC is determined as shown by the example in the FIG.

TABLE 3

| | | | Ave. | |
|---|---|---|---|---|
| | Activity | | CMC | |
| Sample | (cat. | Structure | N = 3 | Min ST |
| Name | SO3) | 2/1 mixture | (PPM) | mN/m |
| C12 Glycerol Acetal Sulfates | 90.9% | 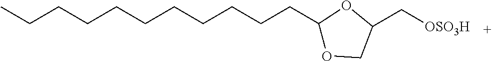 | 323 | 34.7 |
| | | 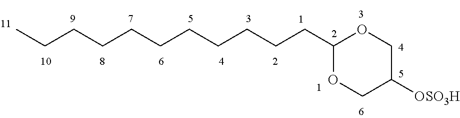 | | |

### EXAMPLE 1

### Preparation of Dodecanal Glyceryl Acetal Sodium Sulfate:

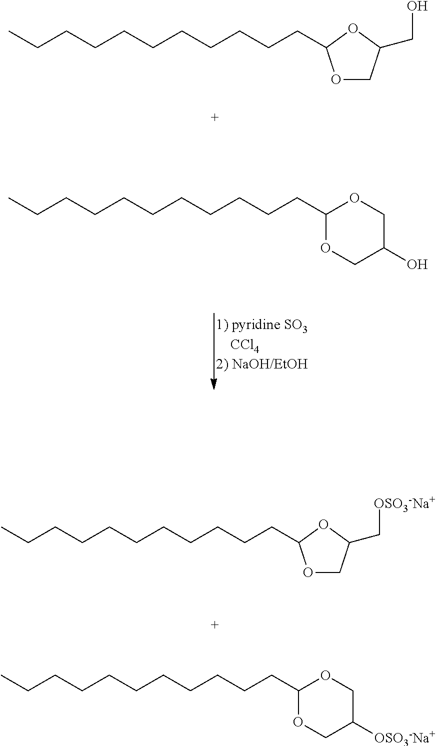

To a 500-ml, single neck, round bottom reaction flask equipped with a magnetic stir bar was added 62.622 grams (0.2423 moles) of Dodecanal Glyceryl Acetal (which is a mixture of isomers of cis- and trans-2-undecyl-1,3-dioxan-5-ol, and cis- and trans-(2-undecyl-1,3-dioxolan-4-yl)methanol, 150 ml of Carbon Tetrachloride and 1 ml of Pyridine. With mixing at room temperature, 98% Sulfur Trioxide Pyridine Complex was added in portions of 10.674 grams, 10.666 grams, 10.948 grams and 9.097 grams for a total of 41.385 grams (equal to 40.56 grams of Sulfur Trioxide Pyridine Complex on 100% basis, 0.2548 moles). Attached to the reaction flask was a water cooled condenser equipped with a nitrogen line at the top leading to a gas bubbler With mixing under a nitrogen atmosphere, the reaction flask was heated for 15 hours at 47° C. using an oil bath. The reaction mixture was concentrated by evaporating off Carbon Tetrachloride under vacuum using a rotary evaporator yielding an off-white solid product, which was then dissolved in a solution prepared from 23.487 g of 50% Sodium Hydroxide, 250 ml of Deionized Water and 250 ml of Absolute Ethanol to form a clear orange solution. The solution pH was measured to be 7-8 using pH test strip paper. To the solution, with mixing, was added enough 6.75 wt % Sodium Hydroxide Solution to bring the solution to pH 10-11. The solution was transferred to a 2-L separatory funnel and extracted twice with Hexanes solvent The water/ethanol layer was isolated and concentrated under vacuum using a rotary evaporator (while heating with a 40° C. water bath) until the product began to foam excessively. Concentrating was stopped, absolute ethanol was added to the product and concentrating was resumed until product again began to foam excessively. This process of absolute ethanol addition followed by additional concentration was repeated until a very viscous solution was obtained as observed with the with human eye which was then transferred to a large crystallizing dish, the dish was partially covered with a watch glass, and a nitrogen stream was blown over the surface of the product solution overnight. The next day, the product crystallized and was placed in a vacuum oven under full vacuum (0.75 mm Hg) at room temperature. After 3 days in the vacuum oven, the product was a crisp solid, which was ground into a powder using a mortar and pestle and placed back into the vacuum oven under full vacuum (0.75 mm Hg) at room temperature. After 2 additional days in the vacuum oven, the product was removed and transferred to a bottle for storage. 70.85 grams of a tan, powered product was recovered.

The final active level of the Dodecanal Glyceryl Acetal Sodium Sulfate product was determined to be 77.22% by Cationic SO3 colorimetric two-phase (water/chloroform) mixed indicator (Dimidium Bromide/Patent Blue VF) titration method using Hyamine 1622 as the cationic titrant as described in Method ASTM D3049— 89-Standard Test Method for Synthetic Anionic Ingredient by Cationic Titration and as described by Reid V. W., G. F. Longman, E. Heinerth, “Determination of anionic active detergents by two-phase titration”, Tenside 4, 292-304 (1967).

### EXAMPLE 2

### Preparation of Dodecanal Glyceryl Acetal Diethyl Ether:

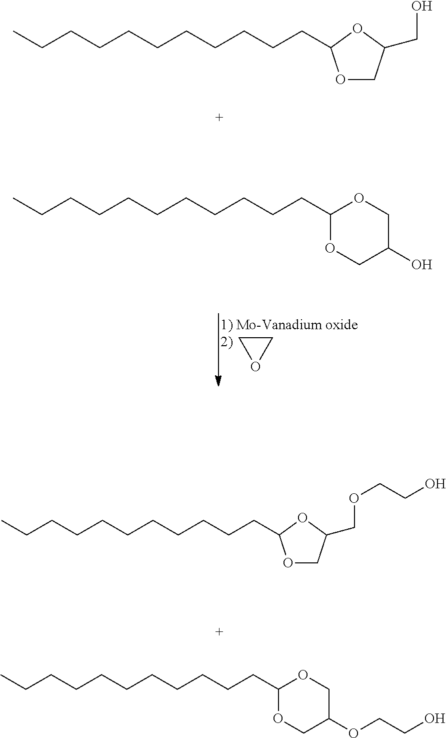

This procedure is a modification of that reported by Zhang, X.; et. al.; “Epoxide hydrolysis and alcoholysis reactions over crystalline Mo-V-oxide”; 2016, RSC Advances, 6 (75), p. 70842-70847. To a 15 mL pressure vessel was added 0.01g Mo-V-oxide (CAS 12209-58-4) and 1 mL of a 4:1 H20/DMF mixture, followed by the alcohol mixture and ethylene oxide gas (3 mmol each). The reaction was heated at 100° C. for 12 hr, then cooled to room temperature (RT). The product was extracted out using EtOAc in a separatory funnel, then purified by running through a column of silica gel. The result was a clear oil.

### EXAMPLE 3

### General Preparation of Dodecanal Glyceryl Acetal Polyethers

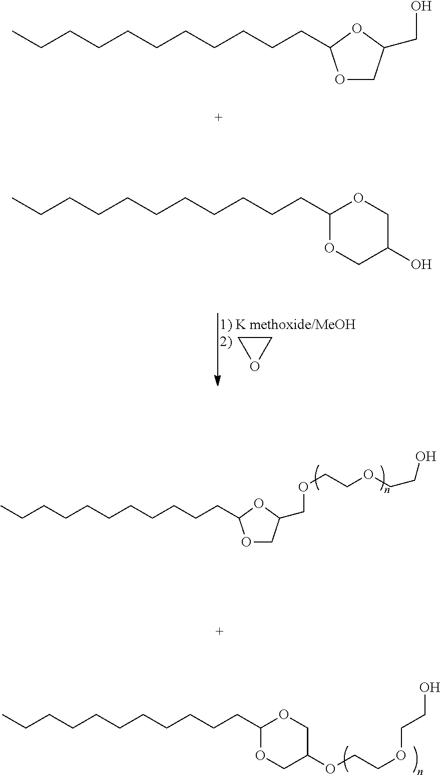

The ethoxylation reactor used was a Model Number 4572 Parr 1800 ml reactor constructed of T316 stainless steel (Parr Instrument Company, Moline, Ill.). It has a Magnetic Drive stirring assembly that uses an electric motor for agitation. The stir shaft has 2 each pitched blade impellers. The reactor has a cooling coil and water is used in the cooling coil to keep the temperature from exceeding a programmed setpoint. The reactor is monitored and controlled by a Camile data acquisition and control system along with the connected automated control valves and other devices.

306.40 g of Dodecanal Glyceryl Acetal composition from Example 1 was added to the reactor along with 5.01 g of 24.90 wt % active Potassium Methoxide solution in methanol. The reactor was purged of air using vacuum and nitrogen cycles. Methanol was removed by sparging with nitrogen. This was done by adding a trickle of nitrogen through the drain valve located on the bottom of the reactor while using a water aspirator for a vacuum source and adjusting the reactor temperature to 100° C.-110° C. and while keeping the reactor pressure below −0.08 MPa by adjusting the nitrogen flow rate. After 2 hours the nitrogen sparge was stopped and the reactor was filled with nitrogen from above and then vented off to ˜0 MPa. The reactor was closed off and then heated to between 110° C. and 125° C. with the agitator stir rate adjusted to 250 rpm (used throughout). 366.00 grams of Ethylene oxide was slowly added to the reactor using automated control valves. The addition of ethylene oxide causes the reactor temperature to increase but this was managed by automated cooling water while controlling the rate at which the ethylene oxide was added. The total pressure was kept below 1.38 MPa until all the ethylene oxide was added. After ethylene oxide addition was complete, the pressure from the ethylene oxide slowly drops as it was consumed by the reaction and eventually the pressure levels off. Once the pressure levels off and was constant for ˜30 minutes, the reaction was deemed to be complete and was ready for sampling.

Any residual ethylene oxide was removed by sparging with nitrogen while using a water aspirator for a vacuum source. During this procedure, the reactor temperature was kept at —110° C. and the reactor pressure was kept below −0.08 MPa. After 30 minutes, the reactor was cooled to below 80° C. and a 320.00 g sample of Dodecanal Glyceryl Acetal 7 Mole Ethoxylate was drained from the reactor to a glass jar while keeping the sample blanketed with low pressure nitrogen. The reactor was closed off after collection of the sample.

Based on mass balance calculations, 354.20 g of Dodecanal Glyceryl Acetal 7 Mole Ethoxylate (n=6) remains in the reactor.

The reactor was heated to between 110° C. and 125° C. with the agitator stir rate adjusted to 250 rpm (used throughout) and 55.00 grams of Ethylene oxide was slowly added to the reactor using automated control valves. The addition of ethylene oxide causes the reactor temperature to increase but this was managed by automated cooling water while controlling the rate at which the ethylene oxide was added. The total pressure was kept below 1.38 MPa until all the ethylene oxide was added. After ethylene oxide addition was complete, the pressure from the ethylene oxide slowly drops as it was consumed by the reaction and eventually the pressure levels off. Once the pressure levels off and was constant for ˜30 minutes, the reaction was deemed to be complete and was ready for sampling.

Any residual ethylene oxide was removed by sparging with nitrogen while using a water aspirator for a vacuum source. During this procedure, the reactor temperature was kept at ˜110° C. and the reactor pressure was kept below -.08MPa. After 30 minutes, the reactor was cooled to below 80° C. and based on mass balance calculation, 407.40 g of Dodecanal Glyceryl Acetal 9 Mole Ethoxylate was contained in the reactor for drainage to a glass jar while keeping the sample blanketed with low pressure nitrogen.

By NMR analysis, the desired Dodecanal Glyceryl Acetal 7 Mole Ethoxylate and Dodecanal Glyceryl Acetal 9 Mole Ethoxylate (n=8) were obtained.

Test Solutions were prepared at 359 mg/L (ppm) at a weight ratio of anionic surfactant to C12,14 dimethyl amine oxide of 3.7:1. The anionic surfactant was varied at ratios of 100:0, 75:25, 50:50, 25:75, and 0:100 of C12,14 alkyl sulfate, Sodium salt: Dodecanal Glyceryl Acetal Sodium Sulfate (from Example 1). C12,14 alkyl sulfate, Sodium salt was commonly used as anionic surfactant within liquid hand dishwashing detergent formulations, single variably replacing the sodium dodecyl glycerol acetal sulfate anionic surfactant within the examples according to the invention.

Suds Generation, Suds Mileage, and Minimum Interfacial Tension Values for the test solutions were measured according to the methods described above. The data tabulated below clearly shows the improved suds mileage performance in the presence of soil while demonstrating strong suds generation and improved grease handling potential as demonstrated by the lower oil/water interfacial tensions.

Materials Used:
Sodium dodecyl glycerol acetal sulfate (C12-GAS) from Example 1
C12-14 dimethyl Amine Oxide (C12,14 DMAO)
C12 14 alkyl sulfate, Sodium salt (C12,14 AS)

TABLE 4
Suds Generation, Suds Mileage, and Minimum Interfacial Tension
Values for the surfactant mixtures according to the invention.

| | C12, | C12 | C12,14 | Total | Suds | Suds | |
|---|---|---|---|---|---|---|---|
| | 14 AS | GAS | DMAO | Surfactant | Generation | Mileage | Minimum |
| | mg/L | mg/L | mg/L | mg/L | Index | Index | IFT, mN/m |
| Example | 282.6 | 0 | 76.4 | 359 | 100 | 100 | 0.44 |
| 1 | | | | | | | |
| Example | 212 | 70.6 | 76.4 | 359 | 98 | 119 | 0.18 |
| A | | | | | | | |
| Example | 141.3 | 141.3 | 76.4 | 359 | 96 | 106 | 0.24 |
| B | | | | | | | |
| Example | 70.6 | 212 | 76.4 | 359 | 91 | 104 | 0.34 |
| C | | | | | | | |
| Example | 0 | 282.6 | 76.4 | 359 | 87 | 83 | 0.59 |
| 2 | | | | | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, comprising any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

The following is claimed:

1. A compound comprising the following structure: 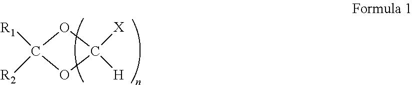
where:
n=2 or 3;
X=—H or -A-B such that only one carbon atom of the —(CH—X)ₙ— linking chain is attached to -A-B;
A=—O— or —CH₂—;
B=—SO₃⁻M⁺, where is M is a cation that forms an acceptable salt, —SO₃H, or —R₃ only when A═—O—; Ri=a substituted or unsubstituted straight chain or branched chain alkyl or alkenyl, having 1 to 22 carbon atoms;
R₂=H;
R₃=hydroxyethyl, hydroxypropyl, methyl hydroxyethyl;
including all possible stereoisomers thereof.

2. The compound of claim 1 comprising at least one of a sulfate and sulfonate, wherein the sulfate is at least one of:
(2-(heptan-3 -yl)-1,3-dioxan-5-yl)methyl hydrogen sulfate;
(2-(nonan-4-yl)-1,3-dioxan-5-yl)methyl hydrogen sulfate;
(2-dodecyl-1,3-dioxan-4-yl)methyl hydrogen sulfate;
(2-decyl-1,3-dioxolan-4-yl)methyl hydrogen sulfate;
(2-(dodecan-2-yl)-1,3-dioxan-4-yl)methyl hydrogen sulfate;
(2-(dodecan-3-yl)-1,3-dioxan-4-yl)methyl hydrogen sulfate;
(2-(dodecan-4-yl)-1,3-dioxan-4-yl)methyl hydrogen sulfate;
(2-(dodecan-6-yl)-1,3-dioxan-4-yl)methyl hydrogen sulfate;
(Z)-(1-phenylhex-1-en-2-yl)-1,3-dioxolan-4-yl)methyl hydrogen sulfate;
(Z)-2-heptadec-8-en-1-yl)-1,3-dioxolan-4-yl)methyl hydrogen sulfate;
stereoisomers thereof, salt forms thereof, or mixtures thereof.

3. The compound of claim 1 comprising at least one of a sulfate and sulfonate, wherein the sulfate or sulfonate is at least one of:
(2-(heptan-3-yl)-1,3-dioxolan-4-yl)methyl hydrogen sulfate;
(2-(nonan-4-yl)-1,3-dioxolan-4-yl)methyl hydrogen sulfate;
(2-dodecyl-1,3-dioxolan-4-yl)methyl hydrogen sulfate;
(2-dodecan-2-yl)-1,3-dioxolan-4-yl)methyl hydrogen sulfate;
(2-(dodecan-3-yl)-1,3-dioxolan-4-yl)methyl hydrogen sulfate;
(2-(dodecan-4-yl)-1,3-dioxolan-4-yl)methyl hydrogen sulfate;
(2-(dodecan-5-yl)-1,3-dioxolan-4-yl)methyl hydrogen sulfate;
(2-(dodecan-6-yl)-1,3-dioxolan-4-yl)methyl hydrogen sulfate;
(Z)-(2-(1-phenylhex-1-en-2-yl)-1,3-dioxolan-4-yl)methyl hydrogen sulfate;
(Z)-(2-heptadec-8-en-1-yl)-1,3-dioxolan-4-yl)methyl hydrogen sulfate;
(Z)-(2-heptadec-8-en-1-yl)-1,3-dioxolan-5-yl)hydrogen sulfate;
(2-dodecyl-1,3-dioxolan-4-yl)methyl hydrogen sulfate;
stereoisomers thereof, salt forms thereof, or mixtures thereof.

4. The compound of claim 1 comprising at least one of a sulfate and sulfonate, wherein the sulfate or sulfonate is at least one of:
(2-(heptan-3 -yl)-1,3-dioxolan-4-yl)methyl hydrogen sulfate;
(2-(nonan-4-yl)-1,3-dioxolan-4-yl)methyl hydrogen sulfate;
(2-dodecyl-1,3 -dioxolan-4-yl)methyl hydrogen sulfate;
(2-dodecan-2-yl)-1,3-dioxolan-4-yl)methyl hydrogen sulfate;
(2-(dodecan-3-yl)-1,3-dioxolan-4-yl)methyl hydrogen sulfate;
(2-(dodecan-4-yl)-1,3-dioxolan-4-yl)methyl hydrogen sulfate;
(2-(dodecan-5-yl)-1,3-dioxolan-4-yl)methyl hydrogen sulfate;
(2-(dodecan-6-yl)-1,3-dioxolan-4-yl)methyl hydrogen sulfate;
(Z)-(2-(1-phenylhex-1-en-2-yl)-1,3-dioxolan-4-yl)methyl hydrogen sulfate;
(Z)-(2-heptadec-8-en-1-yl)-1,3-dioxolan-4-yl)methyl hydrogen sulfate;
(Z)-(2-(heptadec-8-en-1-yl)-1,3-dioxolan-4-yl)methanesulfonic acid;
(2-dodecyl-1,3-dioxolan-4-yl)methyl hydrogen sulfate;
stereoisomers thereof, salt forms thereof, or mixtures thereof.

5. The compound of claim 1 comprising an alkoxylate that is at least one of:
2-((2-undecyl-1,3-dioxan-5-yl)oxy)ethan-1-ol;
2-((2-tridecyl-1,3-dioxan-5-yl)oxy)ethan-1-ol;
2-((2-pentadecyl-1,3-dioxan-5-yl)oxy)ethan-1-ol;
1-((2-tridecyl-1,3-dioxan-5-yl)oxy)propan-2-ol;
1-((2-undecyl-1,3-dioxan-5-yl)oxy)propan-2-ol;
3-((2-undecyl-1,3-dioxan-5-yl)oxy)propan-1-ol;
3-((2-tridecyl-1,3-dioxan-5-yl)oxy)propan-1-ol;
3-((2-pentadecyl-1,3-dioxan-5-yl)oxy)propan-1-ol;
20-((2-undecyl-1,3-dioxan-5-yl)oxy)-3,6,9,12,15,18-hexaoxaicosan-1-ol;
26-((2-undecyl-1,3-dioxan-5-yl)oxy)-3,6,9,12,15,18,21,24-octaoxahexacosan-1-ol;
stereoisomers thereof, salt forms thereof, or mixtures thereof.

6. The compound of claim 1 comprising an alkoxylate that is at least one of:
2-((2-undecyl-1,3-dioxolan-4-yemethoxy)ethan-1-ol;
2-((2-tridecyl-1,3-dioxolan-4-yl)methoxy)ethan-1-ol
2-((2-pentadecyl-1,3-dioxolan-4-yl)methoxy)ethan-1-ol;
1-((2-pentadecyl-1,3-dioxolan-4-yl)methoxy)propan-2-ol;
1-((2-tridecyl-1,3-dioxolan-4-yl)methoxy)propan-2-ol;
1-((2-undecyl-1,3-dioxolan-4-yl)methoxy)propan-2-ol;
3-((2-undecyl-1,3-dioxolan-4-yl)methoxy)propan-1-ol;
3-((2-tridecyl-1,3-dioxolan-4-yl)methoxy)propan-1-ol;
3-((2-pentadecyl-1,3-dioxolan-4-yl)methoxy)propan-1-ol;
1-(2-undecyl-1,3-dioxolan-4-yl)-2,5,8,11,14,17,20-heptaoxadocosan-22-ol;
1-(2-undecyl-1,3-dioxolan-4-yl)-2,5,8,11,14,17,20,23,26-nonaoxaoctacosan-28-ol;;
stereoisomers thereof, salt forms thereof, or mixtures thereof.

7. A detergent composition comprising the compound of claim 1.
